(19) United States
(12) Patent Application Publication
(10) Pub. No.: US 2025/0072948 A1
(43) Pub. Date: Mar. 6, 2025

(54) HELICAL ELECTRODE TREATMENT DEVICE AND RELATED METHODS OF TREATMENT

(71) Applicant: Intuitive Surgical Operations, Inc., Sunnyvale, CA (US)

(72) Inventors: Jonathan COPE, Los Gatos, CA (US); William CAMISA, Santa Clara, CA (US); David JOHNSON, Aptos, CA (US)

(73) Assignee: Intuitive Surgical Operations, Inc., Sunnyvale, CA (US)

(21) Appl. No.: 18/818,408

(22) Filed: Aug. 28, 2024

Related U.S. Application Data
(60) Provisional application No. 63/579,822, filed on Aug. 31, 2023.

(51) Int. Cl. A61B 18/00; A61B 34/37

(52) U.S. Cl. CPC A61B 18/00; A61B 34/37; A61B 2018/00077; A61B 2018/00083; A61B 2018/00196; A61B 2018/00202; A61B 2018/00214; A61B 2018/00613; A61B 2018/00904

(57) ABSTRACT

An electrode treatment device includes an electrode strip arranged in the shape of a helix with a first end of the electrode strip attached to an elongate flexible body, such as a catheter tube, and a second end of the electrode strip attached to a flexible shaft that extends from the body. When the helical electrode is contracted around the body and/or shaft, the device may be less than 2 mm in diameter to facilitate endoluminal insertion and treatment of gastrointestinal or lung tissue, for example. The shaft is rotatable relative to the body to radially expand the helix to a deployed position in apposition to target tissue for electrosurgical treatment (e.g., treatment in a lumen). The electrode strip may consist of conductive foil or may comprise a flexible printed circuit with one or more electrodes formed thereon. The device may be operated by a robotic manipulator.

### CROSS-REFERENCE TO RELATED APPLICATION

This application claims priority benefit under 35 U.S.C. § 119 (e) of U.S. Provisional Patent Application No. 63/579,822, filed Aug. 31, 2023, which is hereby incorporated by reference in its entirety.

### TECHNICAL FIELD

The present disclosure relates generally to the field of tissue treatment. In particular, the disclosure relates to devices and methods with deployable electrodes to ablate cells for treating targeted tissues.

### BACKGROUND

Tissues in the body may be treated in surgical or medical procedures using resection and removal. While such procedures may be necessary to treat more extensive damage or resolve various medical conditions, minimally invasive procedures may be used as an alternative in some situations. Such procedures, where applicable, may be preferable to reduce overall patient risk, damage to surrounding tissue, recovery time, discomfort, and potentially harmful side effects as compared to more invasive, surgical procedures. In minimally invasive tissue ablation, targeted tissues may be treated inside the body (e.g., in situ) using procedures that do not involve resection or may require minimal resection. Some examples of minimally invasive tissue ablation techniques include electrolytic ablation, cryosurgery, chemical ablation (e.g., alcohol injection), thermal ablation (e.g., radiofrequency, microwave), and hydrothermal ablation. The primary aim of these ablation procedures is to destroy abnormal tissue in a targeted region and encourage the regrowth of healthy tissue.

Another known minimally invasive treatment technique involves electroporation of targeted tissues by localized application of an electrical field to increase permeability of cell membranes, which may then allow drugs or other chemicals to be introduced into the cells for treatment. Electroporation may also be used in combination with electrolysis as a method of tissue ablation, by a process also known as electrolytic electroporation, electroporation-electrolysis, or E2.

Tissue ablation is a therapeutic procedure used to treat abnormal or damaged tissues of various types, such as those of the gastrointestinal tract. Damage to intestinal tissue may arise from several sources but is commonly associated with chronic metabolic disorders such as diabetes. For patients with diabetes, damage to gastrointestinal tissues (particularly the duodenum) may create issues with insulin resistance and/or impair the ability for the body to process glucose. To help improve patient management of the disorder, duodenal mucosal resurfacing (DMR) has been proposed to resurface the patient's intestinal lining and help regenerate healthy lining to improve nutrient absorption at the duodenum. Improved health of the intestinal lining helps correct absorption issues and may yield a better therapeutic response to insulin for the patient, which in turn may allow some patients to replace more aggressive insulin therapies (e.g., injections) with oral medications to help manage the condition.

Generally, DMR is a procedure that involves introducing a catheter (or other suitable medical instrument) into the duodenum, typically under endoscopic guidance, and ablating the inner lining of the duodenum. However, various ablation techniques and ablation instrument designs may not be suitable for endoluminal ablation or may be challenging to control ablation to desired depths via application from within the duodenum lumen.

Tissue ablation also has potential as a minimally invasive therapeutic procedure for targeted treatment of lung cancer. However, the present inventors have recognized that deployment of electrodes for electrolysis and/or electroporation is complicated by size constraints and guidance issues associated with navigating a catheter through the tree of relatively small bronchioles leading to targeted lung tissues. Navigation of the bronchiole tree may require a steerable catheter having a diameter less than 3.5 mm. The present inventors have recognized the challenges associated with designing an electrode array small enough to be carried by such a catheter, but which can be deployed or expanded at the treatment site to contact a larger area of tissue.

The inventors have identified a need for an improved medical instrument with a flexible substrate having one or more electrodes to ablate cells for treating targeted tissues, such as gastrointestinal tissues or lung tissues for example.

### SUMMARY

An electrode treatment device includes an elongate body, such as a catheter tube, having a distal end portion terminating in a tip, a shaft rotatably supported by the body and extending beyond the tip of the body, and an electrode strip, such as a flexible printed circuit with one or more electrodes extending along its length. The electrode strip has a first end portion attached to the distal end portion of the body and a second end portion attached to the shaft at a location distal to the tip of the body, and is wound around the body and/or the shaft to form a helix. The electrode strip may be coupled to an electrical power source via wires or other conductive pathways. The shaft is rotatable in a first direction relative to the body for radially expanding the helix to thereby deploy the electrode strip at a treatment site into apposition with targeted tissue for performing an electrosurgical procedure such as DMR or another function. The shaft is also rotatable relative to the body in a second direction opposite the first direction for radially contracting the helix around the body and/or shaft to facilitate extraction of the electrode strip from the treatment site. In some embodiments, the shaft is also slidably movable inwardly and distally outwardly relative to the body to adjust a pitch of the helix during expansion (deployment) and/or contraction.

In some embodiments, the electrode strip may be arranged to leave no gaps between turns of the helix when the electrode strip is contracted around the body and/or the shaft, or when in the expanded (deployed) position. And in some embodiments, the electrode strip is thermoformed or heat treated so that the electrode strip tends to return to the shape of the helix when no force is applied to the electrode strip via the shaft and/or the body.

In some embodiments, the shaft is movable relative to the body to cause the electrode strip to move between the contracted position and a maximally expanded position and the electrode strip has a maximum expansion ratio that exceeds 5:1 or 10:1 or 20:1, or is in the range of 5:1 to 30:1.

In some embodiments, the electrode treatment device is operated by a robotic manipulator. The robotic manipulator may include a rotational actuator and a translational actuator, which may be independently controlled in some embodiments. In some embodiments, the rotational and translational actuators are integrated into a single actuator.

Additional aspects and advantages will be apparent from the following detailed description of preferred embodiments, which proceeds with reference to the accompanying drawings.

### BRIEF DESCRIPTION OF THE DRAWINGS

The drawings depict only several examples in accordance with the disclosure and are therefore not to be considered limiting in scope. Exemplary embodiments will be described with additional specificity and detail through use of the accompanying drawings, in which:

FIG. 1 is a schematic illustration of a system for electroporation and/or electrolysis in accordance with examples described herein;

FIG. 2 is a schematic illustration of a robotically-assisted manipulator system in accordance with examples described herein;

FIG. 3A is a schematic diagram of an instrument system in accordance with examples described herein;

FIG. 3B illustrates a distal portion of the instrument system of FIG. 3A with an extended example of an instrument in accordance with examples described herein;

FIG. 4 illustrates an electrode instrument with a helical electrode strip in a contracted position, accordance with an example described herein;

FIG. 5 illustrates the electrode instrument of FIG. 4 with the helical electrode strip deployed to an expanded position;

FIGS. 6A, 6B, and 6C schematically illustrate an electrode instrument according to examples described herein, in which a body and shaft of the instrument are both rotatable and slidably translatable relative to each other to control a pitch of the helical electrode; and

FIGS. 7A, 7B, and 7C illustrate incremental advancement of a helical electrode along a treatment site to a series of overlapping contact areas or treatment regions.

### DETAILED DESCRIPTION

This disclosure is directed to apparatuses and systems for providing controlled delivery of one or more flexible electrodes to a targeted tissue site for treatment, such as ablation by electroporation and/or electrolysis, or for other purposes. Generally, a method for minimally invasive regenerative surgery is disclosed which includes subjecting a target area in living tissue to ablation delivered via the one or more electrodes. In some examples, the ablation energy may be in the form of a combination of one or more electric fields and electrolysis. However, it should be appreciated that the example systems and methods described herein may be utilized for the deployment of flexible substrates carrying electrodes using ablation modalities other than electroporation and/or electrolysis (e.g., radiofrequency ablation), or for purposes other than ablation, such as electrical stimulation or diagnostic methods.

During an example procedure utilizing electroporation and electrolysis, the electrode(s) are brought into proximity and/or contact with the target tissue, and the electric fields are generated by applying voltages and/or currents between the one or more electrodes. The electric fields may be generated to have a magnitude to permeabilize cell membranes in a region where ablation is desired. The electric fields may be generated to produce products of electrolysis of a magnitude that, by themselves, do not cause damage to cells or the extracellular matrix located in and around the target treatment region. However, when sufficient products of electrolysis are generated in the region of permeabilized cells, cellular death occurs within the region of the applied electric field without damaging the extracellular matrix or scaffolding to aid in promoting tissue regrowth in the treated tissue region.

In some examples, after applying ablative energy to one target region, the electrode(s) may be moved to other tissue locations by advancing and/or retracting a delivery catheter carrying the electrode(s) and repeating the process. In this manner, electroporation and electrolysis may be performed at multiple locations in a patient to cover a larger area of tissue. In some examples, multiple sets of electrodes may be positioned at multiple respective tissue sites such that electroporation and electrolysis may be performed at each site in parallel, reducing and/or eliminating a need to repeat the procedure as a catheter is moved through the patient.

In some embodiments of the present disclosure, the systems and methods disclosed herein may be used to treat tissue of the gastrointestinal tract. In other embodiments, lung tissue or any of a variety of other tissue may be treated using the systems and methods described herein. Generally, tissue may be treated where tissue regeneration is desirable or where it is desirable to replace one type of cells with another. Examples include intestine, duodenum, stomach, bladder, uterus, endometrial lining, ovaries, colon, rectum, sinuses, ducts, ureters, prostate, skin, muscle, nerve, diaphragm, momentum, kidney, follicles, brain, lymphatic vessels, breast, esophagus, lung, liver, kidney, lymph nodes, lymph node basins and/or heart. Replacement of one type of tissue with another may be in fibrotic areas where it is desired to replace fibrotic cells with stem cells that can remodulate the area or when pancreatic islets are injected in part of the liver to generate new sources of insulin. Other tissue may be treated in other examples. The following provides additional details relating to example processes of electroporation and/or electrolysis for tissue treatment in accordance with some embodiments.

As noted above, electroporation may be performed to permeabilize the cell membranes of targeted cells. Reversible electroporation may be used in which the permeabilization may cease after the electric fields are removed. Cells may survive reversible electroporation with the pores within the membrane resealing and returning to homeostasis. In irreversible electroporation, the permeabilization of the cell membrane is permanent leading to cell death. Typically for biological tissues, electric fields lower than about 1500 V/cm to about 200 V/cm are considered to produce reversible electroporation, while electric fields higher than about 1500 V/cm are considered to produce irreversible electroporation. Examples of systems and methods described herein may utilize reversible electroporation to avoid disadvantages of irreversible electroporation, which may include heating and thermal damage, complexity of providing such large electric fields, and muscle contractions which may result from the large electric fields. It is to be understood that, although systems and methods described herein may be designed to utilize reversible electroporation, there may occur localities or incidental areas where conditions are such that irreversible electroporation, or even thermal ablation, may occur in limited parts of the treated tissue.

In some treatment procedures, products of electrolysis may be applied to permeabilized cells to cause cell death of the permeabilized cells within the applied electrical field while leaving the extracellular matrix of the permeabilized cells intact. The extracellular matrix generally refers to a three-dimensional network of proteins and/or other molecules (e.g., collagen fibers, proteoglycans, and/or proteins such as fibronectin and/or laminin) which provide structure for cells and tissues and may additionally provide signaling for cell growth and development. Extracellular matrices may be used as scaffolds for tissue regeneration and/or engineering. For example, cells may be regenerated, grown, transplanted, or otherwise nurtured, on the extracellular matrix. By retaining the extracellular matrix in regions of otherwise ablated cells described herein, cell regeneration and/or tissue engineering may occur in the region of ablated cells. In some procedures, the extracellular matrix may be transplanted from a region of ablated cells to another region (which may be another region of ablated cells), to promote regeneration and/or tissue engineering in the transplanted region. In some cases, once the extracellular matrix is present, material may be injected into the extracellular matrix to enhance regrowth.

Example systems and methods described herein may utilize electrolysis products to cause cell death together with electroporation to permeabilize the cell membrane. The electrolysis products are sufficient to ablate (e.g., cause cell death) permeabilized cells in a relatively short time frame. However, the concentration of electrolysis products and exposure time are insufficient to cause cell death of non-permeabilized cells, thereby minimizing or avoiding the formation of scar tissue, fibrotic tissue, or ulceration in the treated region. Scar tissue and fibrosis is an indication that the extracellular matrix has been affected, and the ability for cells to regenerate and/or tissue engineering to occur in the ablated region may be inhibited. Accordingly, the electrolysis products are used to ablate permeabilized cells only, while leaving the extracellular matrix intact to promote tissue regrowth. Electrolysis products may include products toxic to cells (cytotoxic). Electrolysis preferentially utilizes one or more inert electrodes that do not participate in the process of electrolysis except as a source or sink of electrons or as catalysts. When participating non-inert electrodes are used in the process, they can generate metal ions that may cause systemic damage to the body, such as excess of iron or even metallic fragments.

Examples of systems disclosed herein include one or more electrodes, a power supply, and a controller to apply energy for treating internal tissue. The energy applied may be for electrolysis and/or electroporation in some examples, radiofrequency ablation in other examples, and other energy modalities in yet other examples. When the energy is for electroporation with or without electrolysis, the controller controls a charge delivered to the electrode(s) to induce one or more electric fields. Where electroporation and electrolysis are included, the electrode(s) are used to generate a current to produce electrolysis products and a voltage difference to produce an electric field that induces electroporation. The duration and magnitude of the charge applied determines the dose of the electrolytic products and the degree of the permeabilization of cells in the treatment site. Accordingly, a region of cell ablation may be determined by a region in which cells are exposed to the combination of permeabilization and to electrolysis products that cause ablation. The ablation, however, may leave the extracellular matrix intact in the region of ablated cells where the electrical field has been applied. The composition of the electrode(s) may be chosen in accordance with the desired products produced and electroporation effects.

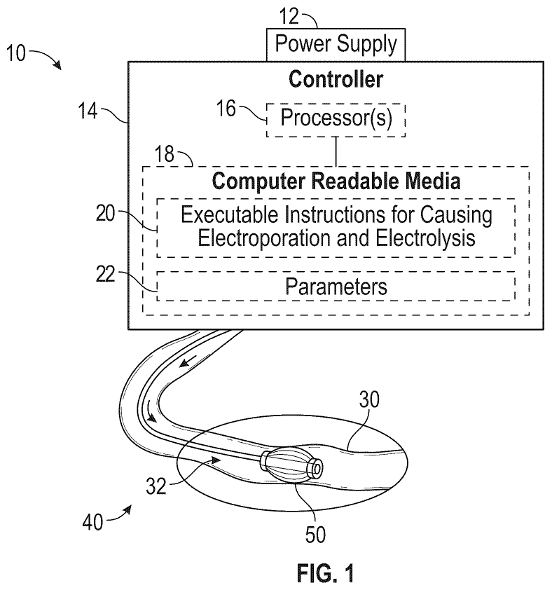

With reference to FIG. 1, the following provides a brief overview of an example system for delivering a catheter with electrodes to a targeted tissue site for treatment via electroporation and/or electrolysis. For clarity, the written description and the associated figures of various example embodiments generally references a catheter for use with the systems and methods described herein. However, it should be understood that other suitable medical instruments (e.g., instruments that may not be specifically classified as catheters) and other suitable energy modalities (e.g., radiofrequency ablation) may be used in conjunction with the disclosed systems and methods without departing from the principles of the disclosed subject matter.

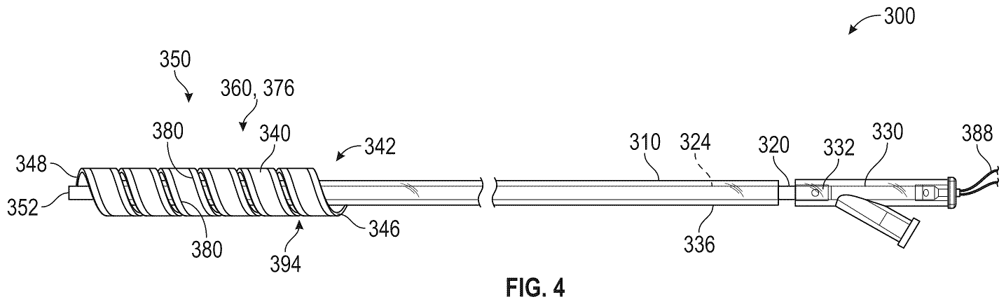

FIG. 1 is a schematic illustration of a system 10 arranged in accordance with example embodiments described herein. Generally, example systems described herein may include a delivery system and a controller. In the example of FIG. 1, the system 10 includes a power supply 12 coupled to a controller 14, and a catheter 40. Examples of other suitable catheters which may be used with the system 10 are described in further detail below with respect to FIGS. 4-7. With reference to FIG. 1, the controller 14 may include a processor 16, computer readable media 18, and other computing system components, such as one or more input devices, output devices, sensors, and/or communication devices in some examples. Additional, fewer, and/or different components may be used in other examples. The computer readable media 18 includes executable instructions 20 for modulating the output of power supply 12 for causing electroporation and electrolysis with the catheter 40 and may include stored parameters 22 which may be used in the process for causing electroporation and electrolysis, such as electric field strengths, voltage and/or current levels, waveform shapes, exposure duration parameters, and any other suitable parameters. In the example of FIG. 1, the circle depicted around an end of the catheter 40 may indicate a region of cells that may be permeabilized via an applied electric field.

The controller 14 may be implemented using a computing device. Examples of computing devices include controllers, microcontrollers, computers, servers, medical devices, smart phones, tablets, wearable devices, and the like. The computing device may be handheld and may have other uses as well. The controller 14 may include one or more processors, such as the processor 16. Any kind or number of processors may be present, including one or more central processing unit(s) (CPUs) or graphics processing unit(s) (GPUs) having any number of cores, controllers, microcontrollers, and/or custom circuitry such as one or more application specific integrated circuits (ASICs) and/or field programmable gate arrays (FPGAs).

The controller 14 described herein may include computer readable media 18, such as memory. Any type or kind of memory may be present (e.g., read only memory (ROM), random access memory (RAM), solid state drive (SSD), secure digital card (SD card), and the like). While a single box is depicted as the computer readable media 18 in FIG. 1, any number of computer readable media 18 devices may be present. The computer readable media 18 may be in communication with (e.g., electrically connected to) the processor 16.

The executable instructions 20 for electroporation and electrolysis may include instructions to control a charge delivered to electrodes, such as electrodes 50 of the catheter 40. Accordingly, the controller 14 may induce a voltage difference across the targeted tissue to generate an electric field that causes permeabilization of cells in an area of tissue targeted for treatment. The electrodes 50 are illustrated in a cavity or lumen 32 formed within tissue 30. Although the catheter 40 is shown disposed within a cavity 32 of tissue 30, the catheter 40 may be on the surface of the tissue 30, inside the tissue 30, and/or proximate to the tissue 30. Moreover, although a catheter 40 is shown for delivering and arranging the electrodes 50 for permeabilization and/or the generation of electrolysis products to treat tissue 30, in other embodiments, other suitable delivery systems may be used.

A controller, such as controller 14 of FIG. 1, may activate one or more selected electrodes 50 to provide an electric field. In some embodiments, the controller 14 may alternate or otherwise select a pattern of activated electrodes 50 (e.g., activating pairs of electrodes in sequence) to shape or deliver a particular electric field. In some examples, fluids or other substances may be injected into, brought into contact with, or otherwise placed in or around the tissue 30 to aid in shaping the electric field generated in the tissue 30. For example, conductive fluids may aid in shaping the field (e.g., by extending the field). In other examples, non-conductive fluids may aid in shaping the field (e.g., by attenuating the field). In some examples, non-conductive fluids or other substances may be injected or otherwise placed in tissue to protect areas where ablation is not desired. The electric field may not penetrate and/or not be carried through the non-conductive fluid, such that the field would not reach tissue where ablation is not desired, or at least be present in insufficient strength to cause permeabilization or other cellular change.

The controller 14 may also be used to induce a current through the tissue 30, such as between electrodes 50, to generate products of electrolysis. The products of electrolysis may cause ablation of the permeabilized cells but are preferably insufficient to destroy the extracellular matrix in the region of the permeabilized cells. The remaining intact extracellular matrix may allow for regeneration of the tissue and tissue engineering as described previously.

In some embodiments, one or more of the electrodes 50 used to apply electroporation may also be used to generate products of electrolysis (in other words, some or all of the electrodes may be used for both electroporation and electrolysis). In other embodiments, a first subset of electrodes 50 used to apply electroporation may be different from a second subset of electrodes 50 used to generate products of electrolysis.

In some embodiments, the power supply 12 is integrated with the controller 14. The power supply 12 may be implemented using any suitable power source, such as one or more AC power sources, DC power sources, batteries, and/or waveform generators. The power supply 12 may supply power to the electrodes 50 to generate a voltage and/or current and, therefore, an electric field and/or electrolysis products in the tissue 30. In some examples, the power supply 12 may be implemented using a signal generator, such as an exponential decay wave generator.

The controller 14 may control the timing, strength, and duration of electric fields and/or electrolysis products provided via the catheter 40. The controller 14 may, for example, be programmed to provide an electronic signal to the catheter 40, via power supply 12, where the electronic signal may be indicative of a dose of treatment, for example, a dose of electrolysis products and/or permeability level of cell. The electronic signal may control the timing and magnitude of the generated electric field, which may allow a user to customize treatment of the tissue 30 as desired. In some embodiments, the controller 14 may include such a program, or include one or more processing devices (e.g., processors), coupled to the computer readable media 18 encoded with executable instructions for electrolysis and permeabilization 20. Although shown as a separate component coupled to the catheter 40 in FIG. 1, in some embodiments, the controller 14 may be integrated as part of the catheter 40. In other embodiments, the controller 14 may include programmable circuitry coupled to the catheter 40 via a wired or wireless connection.

As noted previously, the system 10 may include any suitable parameters 22 for controlling various aspects of the electroporation and electrolysis processes, such as electric field strengths, voltage levels, current levels, waveform shapes, exposure duration parameters, and any other suitable parameters. The parameters 22 may be stored in computer readable media 18 or in other suitable databases in communication with the controller 14. In some embodiments, the controller 14 may be used to calculate the parameters 22, or the controller 14 may be in communication with another system operable to calculate the parameters 22.

In some example embodiments, the parameters 22 for a specific treatment protocol may be determined based on measurements taken in the tissue of interest or from a different sample of similar tissue. For example, measurements may be taken at various voltage levels with particular electrode configurations, and a target voltage level, current, pulse pattern, time constant, and other factors may be identified which cause reversible electroporation and the delivery of electrolysis products to result in cell death of the permeabilized cells as desired.

In some embodiments, examples of parameters 22 which may be used include a delivery of between 1 and 10 voltage pulses between 100 V and 1000 V. Those pulses may be delivered in a system having a capacitance between 50 μF and 100 μF, and a resistance of between 15-20 ohms. A quantity of electrolysis products generated may be related to the delivered charge in Coulombs. There are several ways to calculate the delivered charge. For example, the stored electric charge in a capacitor Q (in coulombs) is equal to the product of the capacitance C (in farads) of the capacitor and the voltage V (in volts) across its terminals. That is, Q=C·V. Also, charge is the product of current I (in amps) and time t (in seconds). That is, I·t=Q. By defining the capacitance and the voltage across the capacitance, the charge may be defined, and accordingly the electrolysis performance determined. When a capacitor is discharged, it generates current and the current multiplied by time must be equal to the charge in the capacitor. When a capacitor is being discharged the current is not constant—it decays exponentially. Therefore, the time measure is given as the exponential decay time constant. The capacitance which controls the time constant is generally obtained from capacitors incorporated in the power supply, such as power supply 12 of FIG. 1.

In some embodiments, the time constant (e.g., exponential decay time constant of the capacitive discharge) may be between 1.7 milliseconds (ms) and 1.8 ms. In other embodiments, the time constant may range from 50 microseconds (μs) to 3 ms. Generally, the lower limit of the time constant is related to a time sufficient to ensure electrolytic species (e.g., products of electrolysis) permeate the targeted area of permeabilized cells. The upper limit of the time constant is generally related to the production of electrolytic species (e.g., products of electrolysis) that can cause ablation on their own. Accordingly, electrolysis is generally targeted for an amount of time sufficient to allow diffusion of electrolysis products through a region of permeabilized cells. However, the amount of time electrolysis is provided should be limited to ensure the process does not result in ablation of non-permeabilized cells or otherwise damages the extracellular matrix in the region of the ablated cells.

In some embodiments, the generated electric field ranges between 100 V/cm and 3500 V/cm. In other embodiments, the electric field may be between 100 V/cm and 1500 V/m or between 200 V/cm and 850 V/cm. In still other embodiments, the electric field may be less than 1400 V/cm in some examples, less than 1300 V/cm in some examples, less than 1000 V/cm in some examples, less than 800 V/cm in some examples, or less than 600 V/cm in some examples.

The system 10 may further include one or more sensors (not shown) for measurement of pH, temperature, electric field strength, tissue electrical resistivity or impedance, and/or other suitable properties of the tissue 30 for optimizing treatment. For example, in one embodiment, a pH sensor may be incorporated with the system 10. The pH sensor may be arranged in any one of several configurations, such as coupled to the catheter 40 adjacent the electrodes 50 to detect a pH value near the electrodes 50. In another embodiment, a pH sensor may be provided at an outer edge of a targeted region of tissue. In either configuration, the pH sensor may be in communication with the controller 14, where the controller 14 may utilize one or more received pH values as an indication of tissue ablation and/or to monitor the occurrence of tissue damage based on detected pH levels at the treatment site and surrounding regions of tissue. In some embodiments, the controller 14 may adjust the voltage, current, and/or electric field applied to the tissue responsive to the detected pH levels. For example, if pH values for tissue located outside the treatment site are at or exceed a threshold for tissue damage, the controller 14 may reduce a magnitude of electric field, a duration between pulses, or cease application of the electric field. Similarly, if pH values for tissue within a targeted region are at or exceed a threshold for tissue ablation, the controller 14 may cease application of current through electrodes immediately and/or after a desired elapsed electrolysis time to cease the electrolysis process.

In some embodiments, a resistivity meter may be used to determine a resistance of the target tissue. For example, the controller 14 and/or power supply 12 of FIG. 1 may provide an impedance measurement. The impedance measurement may determine a resistivity of the tissue 30 contacted by electrodes 50 of the system 10. For example, the controller 14 and/or power supply 12 may provide a nominal amount of current, such as DC current, through the tissue 30 and receive a resistivity measurement and/or calculate resistivity of the tissue 30. In some examples, an applied voltage, current, and/or electric field may be selected, determined, and/or allowed based on a measured resistance of the tissue 30. In some examples, a number of pulses of applied voltage may be selected, determined, and/or otherwise used based on a measured resistance of the tissue.

In some embodiments, a sensor, such as a Gauss meter and/or Tesla meter, for detecting and/or determining electric field strength may also be used. In such embodiments, the sensor may be in operable communication with the controller 14 for ensuring that the electric field strength remains at a target level for a desired treatment protocol.

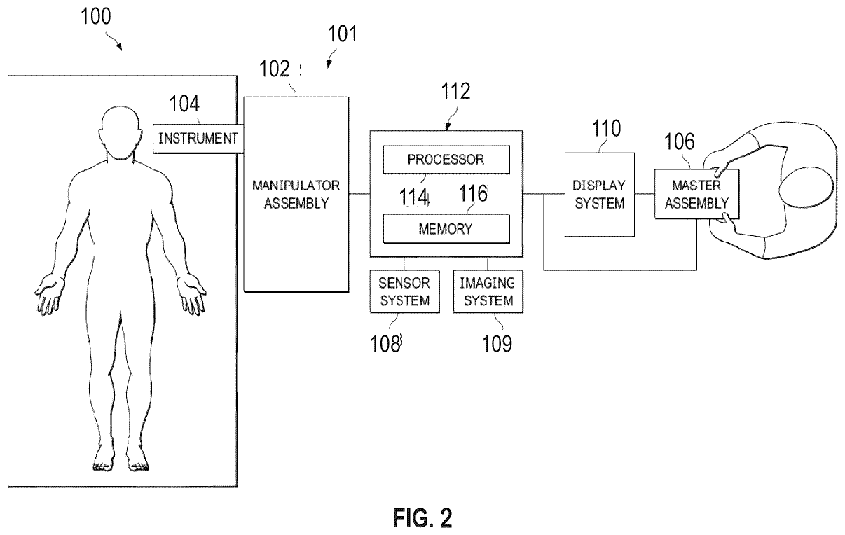

In some embodiments, the ablation instrument for delivering electrolytic electroporation (e.g., catheter 40 of FIG. 1) may be delivered via a computer-assisted, teleoperational manipulator system, sometimes referred to as a robotically assisted system or a robotic system. The manipulator system comprises one or more manipulators that can be operated with the assistance of an electronic controller (e.g., computer) to move and control functions of one or more instruments when coupled to the manipulators. FIG. 2 illustrates one example embodiment of a robotically-assisted manipulator system 100 for use with the systems and methods described herein. The manipulator system can be used, for example, in surgical, diagnostic, therapeutic, biopsy, or non-medical procedures.

With reference to FIG. 2, a robotically-assisted manipulator system 100 may include one or more manipulator assemblies 102 for operating one or more medical instrument systems 104 in performing various procedures on a patient P positioned on a table T in a medical environment 101. For example, the manipulator assembly 102 may drive catheter or end effector motion, may apply treatment to target tissue, and/or may manipulate control members. The manipulator assembly 102 may be teleoperated, non-teleoperated, or a hybrid teleoperated and non-teleoperated assembly with select degrees of freedom of motion that can be motorized and/or teleoperated and select degrees of freedom of motion that may be non-motorized and/or non-teleoperated. An operator input system 106, which may be inside or outside of the medical environment 101, generally includes one or more control devices for controlling manipulator assembly 102. The manipulator assembly 102 supports a medical instrument system 104 and may optionally include a plurality of actuators or motors that drive inputs on the medical instrument system 104 in response to commands from a control system 112. The actuators may optionally include drive systems that when coupled to the medical instrument system 104 advance the medical instrument system 104 into a naturally or surgically created anatomic orifice. Other drive systems can move the distal end of the medical instrument system 104 in multiple degrees of freedom, which may include three degrees of linear motion (e.g., linear motion along the X, Y, Z Cartesian axes) and in three degrees of rotational motion (e.g., rotation about the X, Y, Z Cartesian axes). The manipulator assembly 102 may support various other systems for irrigation, treatment, or other purposes. Such systems may include fluid systems (including, for example, reservoirs, heating/cooling elements, pumps, and valves), generators, lasers, interrogators, and ablation components.

The robotically-assisted manipulator system 100 also includes a display system 110 for displaying an image or representation of the surgical site and the medical instrument system 104 generated by an imaging system 109 which may include an imaging system, such as an endoscopic imaging system. The display system 110 and the operator input system 106 may be oriented so an operator O can control the medical instrument system 104 and the operator input system 106 with the perception of telepresence. A graphical user interface may be displayable on the display system 110 and/or a display system of an independent planning workstation.

In some examples, the endoscopic imaging system components of the imaging system 109 may be integrally or removably coupled to the medical instrument system 104. However, in some examples, a separate imaging device, such as an endoscope, attached to a separate manipulator assembly may be used with the medical instrument system 104 to image the surgical site. The endoscopic imaging system 109 may be implemented as hardware, firmware, software, or a combination thereof which interact with or are otherwise executed by one or more computer processors, which may include the processors of the control system 112.

The robotically-assisted manipulator system 100 may also include a sensor system 108. The sensor system 108 may include a position/location sensor system (e.g., an actuator encoder or an electromagnetic (EM) sensor system) and/or a shape sensor system (e.g., an optical fiber shape sensor) for determining the position, orientation, speed, velocity, pose, and/or shape of the medical instrument system 104. The sensor system 108 may also include temperature, pressure, force, or contact sensors or the like.

The robotically-assisted manipulator system 100 may also include a control system 112. The control system 112 includes at least one memory 116 and at least one computer processor 114 for effecting control between the medical instrument system 104, the operator input system 106, the sensor system 108, and the display system 110. The control system 112 also includes programmed instructions (e.g., a non-transitory machine-readable medium storing the instructions) to implement a procedure using the robotically-assisted manipulator system including for navigation, steering, imaging, engagement feature deployment or retraction, applying treatment to target tissue (e.g., via the application of energy), or the like.

The control system 112 may optionally further include a virtual visualization system to provide navigation assistance to operator O when controlling the medical instrument system 104 during an image-guided surgical procedure. Virtual navigation using the virtual visualization system may be based upon reference to an acquired pre-operative or intra-operative dataset of anatomic passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like. The control system 112 may use a pre-operative image to locate the target tissue (using vision imaging techniques and/or by receiving user input) and create a pre-operative plan, including an optimal first location for performing treatment. The pre-operative plan can include, for example, a planned size to expand an expandable device, a treatment duration, a treatment temperature, and/or multiple deployment locations.

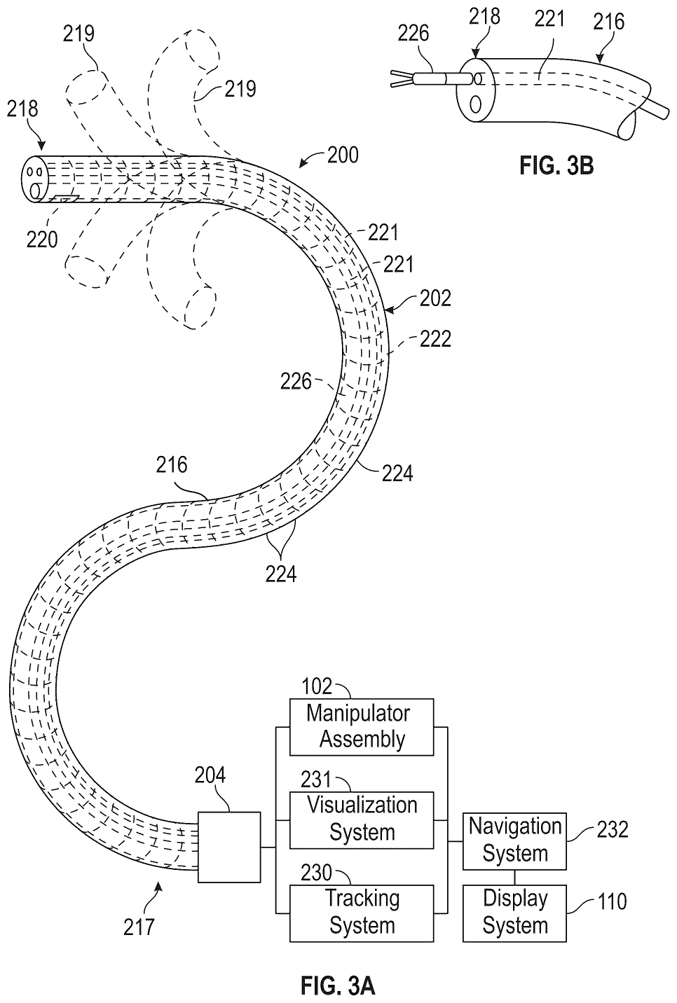

FIG. 3A illustrates a medical instrument system 200 according to some example embodiments. The medical instrument system 200 may be used in an image-guided medical procedure. In some embodiments, the medical instrument system 200 may be used for non-teleoperational exploratory procedures or in procedures involving traditional manually operated medical instruments, such as endoscopy. In other embodiments, the medical instrument system 200 is interchangeable with, or a variation of, the medical instrument system 104 of FIG. 2.

The medical instrument system 200 includes an elongate flexible device 202, such as a flexible catheter, endoscope (e.g., gastroscope, bronchoscope, duodenoscope), or other suitable device coupled to a drive unit 204. The elongate flexible device 202 includes a flexible body 216 having a proximal end portion 217 and a distal end portion 218, including a tip portion. In some embodiments, the flexible body 216 has an outer diameter of approximately 14-20 mm. Other embodiments of the flexible body 216 may have larger or smaller outer diameters. The flexible body 216 may have an appropriate length to reach certain portions of the anatomy, such as the lungs, sinuses, throat, or the upper or lower gastrointestinal region, when the flexible body 216 is inserted into a patient's oral or nasal cavity.

The medical instrument system 200 optionally includes a tracking system 230 for determining the position, orientation, speed, velocity, pose, and/or shape of the distal end portion 218 and/or of one or more segments 224 along the flexible body 216 using one or more sensors and/or imaging devices. The entire length of the flexible body 216, between the distal end portion 218 and the proximal end portion 217, can be effectively divided into segments 224. The tracking system 230 may optionally be implemented as hardware, firmware, software or a combination thereof which interact with or are otherwise executed by one or more computer processors, which may include the processors of the control system 112 in FIG. 2.

The tracking system 230 may optionally track the distal end portion 218 and/or one or more of the segments 224 using a shape sensor 222. In some embodiments, the tracking system 230 may optionally and/or additionally track the distal end portion 218 using a position sensor system 220, such as an electromagnetic (EM) sensor system. In some examples, the position sensor system 220 may be configured and positioned to measure six degrees of freedom, e.g., three position coordinates X, Y, Z and three orientation angles indicating pitch, yaw, and roll of a base point or five degrees of freedom, e.g., three position coordinates X, Y, Z and two orientation angles indicating pitch and yaw of a base point.

The flexible body 216 includes one or more channels (e.g., passageways) sized and shaped to receive one or more medical instruments 226. In some embodiments, the flexible body 216 includes two channels 221 for separate instruments 226, however, a different number of channels 221 may be provided. FIG. 3B is a simplified diagram of an end portion of the flexible body 216 with the medical instrument 226 extended outwardly therefrom according to some embodiments. In some embodiments, the medical instrument 226 may be used for procedures and aspects of procedures, such as surgery, biopsy, ablation, mapping, imaging, illumination, irrigation, or suction. The medical instrument 226 may be deployed through the channel 221 of the flexible body 216 and used at a target location within the anatomy. In other embodiments, the medical instrument may be integrated with flexible body 216, rather than being received in or deployed through a channel of the flexible body 216. The medical instrument 226 can include, for example, image capture devices, biopsy instruments, ablation instruments, catheters, laser ablation fibers, and/or other surgical, diagnostic, or therapeutic tools. Medical tools can include end effectors having a single working member such as a scalpel, a blunt blade, a lens, an optical fiber, an electrode, and/or the like. Other end effectors can include, for example, forceps, graspers, balloons, needles, scissors, clip appliers, and/or the like. Other end effectors can further include electrically activated end effectors such as electrosurgical electrodes, expandable ablation members, transducers, sensors, imaging devices and/or the like. The medical instrument 226 may be advanced from the opening of channel 221 to perform the procedure (e.g., electroporation and/or electrolysis in the present disclosure) and then retracted back into the channel 221 when the procedure is complete. The medical instrument 226 may be removed from the proximal end 217 of the flexible body 216 or from another optional instrument port (not shown) along the flexible body 216. The medical instrument 226 may be used with an image capture device (e.g., an endoscopic camera) also within the elongate flexible device 202. Alternatively, the medical instrument 226 can itself be the image capture device.

The medical instrument 226 may additionally house cables, linkages, or other actuation controls (not shown) that extend between its proximal and distal end portions 217, 218 to controllably bend the distal end portion 218 of the medical instrument 226. The flexible body 216 may also house cables, linkages, or other steering controls (not shown) that extend between the drive unit 204 and the distal end portion 218 to controllably bend the distal end portion 218 as shown, for example, by the broken dashed line depictions 219 of the distal end portion 218. In some examples, at least four cables are used to provide independent “up-down” steering to control a pitch motion of the distal end portion 218 and “left-right” steering to control a yaw motion of the distal end portion 218. In embodiments in which the medical instrument system 200 is actuated by a robotically-assisted assembly, the drive unit 204 can include drive inputs that removably couple to and receive power from drive elements, such as actuators, of the teleoperational assembly. In some embodiments, the medical instrument system 200 can include gripping features, manual actuators, or other components for manually controlling the motion of the medical instrument system 200. The information from the tracking system 230 can be sent to a navigation system 232 where it is combined with information from the visualization system 231 and/or the preoperatively obtained models to provide the physician or other operator with real-time position information.

In some embodiments, the medical instrument system 200 may be guided manually or via the robotically-assisted manipulator system 100 to deliver the medical instrument 226 to a target tissue site for treatment. In some procedures, the selection between a manual or robotic delivery and the approach may be determined based on the medical application. For example, in some gastrointestinal applications, the medical instrument system 200 (or standalone medical instrument 226) may be delivered via a manual or robotic delivery device endoluminally through a trans-oral or trans-anal approach. A trans-abdominal approach with integrated monopolar or bipolar instrumentation, drop in probes or via catheters may also be used. For urological applications, the medical instrument system 200 (or standalone medical instrument 226) may be delivered via a manual or robotic delivery device endoluminally through a trans-urethral approach, trans-perineal, pre-peritoneal or trans-abdominally with integrated bipolar instrumentation, drop in probes or via catheters. Similarly, for gynecological applications, the medical instrument system 200 (or standalone medical instrument 226) may be delivered via a manual or robotic delivery device endoluminally through a trans-vaginal approach, trans-perineal, or trans-abdominally with integrated bipolar instrumentation, drop in probes or via catheters. For hepatobiliary applications, the medical instrument system 200 (or standalone medical instrument 226) may be delivered via a manual or robotic delivery device endoluminally through a trans-oral approach to reach the ampulla or to go externally into the liver via a trans-gastrointestinal wall route or trans-abdominally with integrated bipolar instrumentation, drop in probes or via catheters. For neurovascular applications, the medical instrument system 200 (or standalone medical instrument 226) may be delivered via a manual or robotic delivery device through an endovascular approach or through a keyhole craniotomy with integrated bipolar instrumentation, drop in probes or via catheters. For cardiac applications, the medical instrument system 200 (or standalone medical instrument 226) may be delivered via a manual or robotic delivery device through an endovascular approach or trans-thoracic with integrated bipolar instrumentation, drop in probes or via catheters.

Embodiments of an electrode deployment system and electrode treatment device, illustrated in the context of an electrode instrument 300 for electrosurgery such as electrolysis and/or electroporation, will now be described with reference to FIGS. 4, 5, 6A, 6B, 6C, 7A, 7B and 7C. In some embodiments, electrode treatment devices and electrode deployment systems according to the present disclosure, such as electrode instruments 300, may be received in and/or deployed from a channel of an endoscope, catheter, or any of the other elongate flexible devices 202 described above in reference to FIGS. 3A and 3B. In other embodiments, electrode instruments 300 according to the present disclosure can be integrated with the body of an endoscope, catheter, or other elongate flexible device.

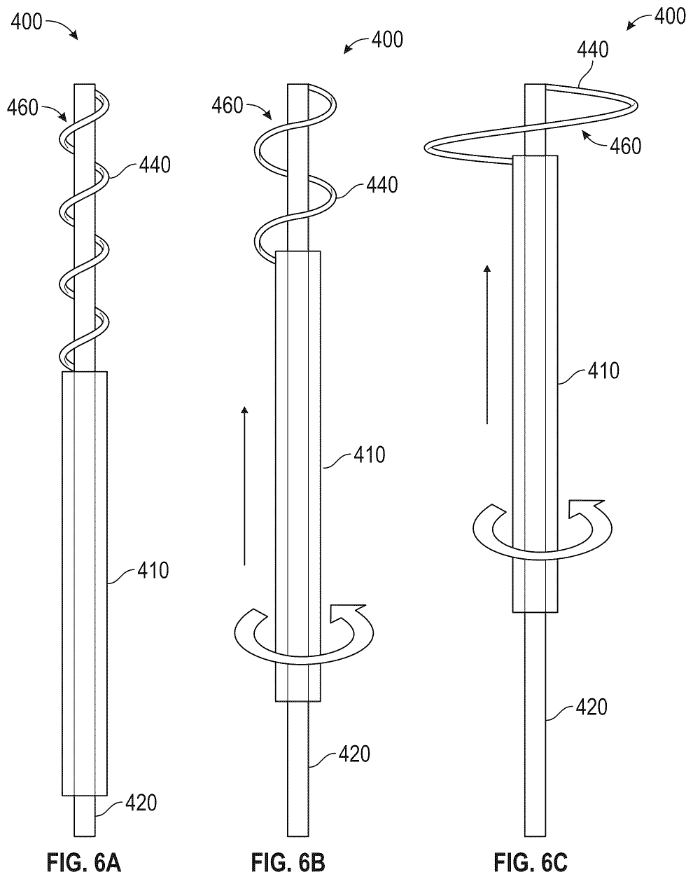

With reference to FIG. 4, electrode instrument 300 includes an elongate body 310 illustrated as a flexible catheter tube, but which may comprise other types of tubular or hollow structures adapted for insertion into a patient, such as a torque tube or an endoscope with a channel or passageway therethrough. A flexible shaft 320, such as a wire, cable, small torque tube, or other drive element, extends through a lumen 324 of body 310 and is movable relative to body 310 rotationally about a longitudinal axis of shaft 320. In some embodiments, shaft 320 is also slidable longitudinally relative to body 310, as described below in reference to FIGS. 6A, 6B, and 6C. A handle 330 is attached to a proximal end portion 332 of shaft 320 to facilitate manipulation of shaft 320. In some embodiments, the handle 330 may be actuated manually by a user. In other embodiments, the handle 330 may be coupled to an external device for actuation such as a robotically-assisted manipulator system. In yet other embodiments, a combination of manual and robotically-assisted actuation may be provided.

The electrode instruments described herein may be coupled to an external device to provide actuation, control, and/or electrical power to the electrode instrument. For example, the electrode instrument 300 and the other electrode instruments described herein may be actuated by a robotically-assisted manipulator system (e.g., manipulator assembly 102). The electrode instrument 300 (e.g., the handle 330, the body 310, and/or the shaft 320) may include or be coupled to a drive unit (e.g., drive unit 204) having one or more drive inputs that removably couple to and receive power from drive elements, such as actuators, of the manipulator assembly. When the electrode instrument 300 is coupled to the manipulator assembly, the drive inputs of the electrode instrument 300 may be coupled to drive outputs of the manipulator assembly that are driven by the drive elements of the manipulator assembly. In addition, the drive inputs of the electrode instrument 300 may be coupled to the distal end portion of the electrode instrument 300 via one or more actuation drive members (e.g., actuation cables, actuation rods, tension members, and the like) to perform actions such as advancing, retracting, or articulating the distal end of the electrode instrument 300 as well as expansion and/or retraction of the electrode strips described herein (e.g. electrode strip 340) to adjust the electrode strip between a deployed position and a contracted position. In some embodiments, the body 310 and/or shaft 320 of the electrode instrument 300 may be coupled to the drive unit 204 for adjusting the electrode strip between the deployed and contracted positions. In addition, the drive unit 204 may be coupled to a controller (e.g., controller 14) and/or power supply (e.g., power supply 12) to provide electrical power to electrodes of electrode instrument 300. The drive unit 204 may be electrically coupled to the electrodes via one or more electrical cables running along or through the electrode instrument 300. In alternative embodiments, the controller and power supply may provide power to the electrical cables without coupling to the drive unit (e.g., the controller and power supply may be independent of the drive unit).

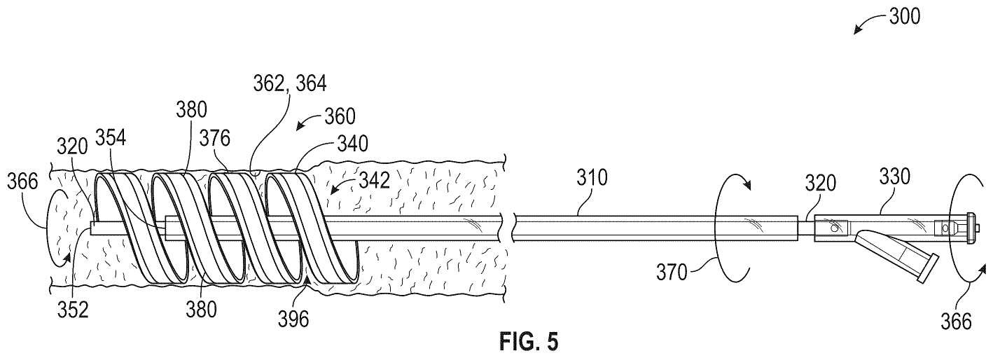

An electrode strip 340 is coiled around a distal end portion 342 of body 310, with a first end portion 346 of electrode strip 340 attached to distal end portion 342 of body 310 and a second end portion 348 of electrode strip 340 attached to a distal end portion 352 of shaft 320 located distal to a tip 354 (FIG. 5) of distal end portion 342 of body 310. The first end portion 346 is a proximal end portion of the electrode strip 340 and the second end portion 348 is a distal end portion of the electrode strip 340. The connection locations of the first and second end portions 346 and 348 of electrode strip 340 are spaced apart longitudinally from each other such that the electrode strip 340 is coiled in the configuration of a helix 360. Helix 360 is formed in a middle portion 376 of electrode strip 340 between first and second end portions 346, 348 which curve away from the middle portion 376 toward their connections to body 310 and shaft 320.

FIG. 4 illustrates the helically-arranged electrode strip 340 in a contracted position or configuration, with electrode strip 340 wrapped closely around body 310 and shaft 320. For clarity of illustration, FIG. 4 shows electrode strip 340 expanded slightly from a fully contracted position wherein the electrode strip 340 is wound tightly around body 310. In the contracted position, electrode instrument 300 is insertable endoluminally into a patient's body for treating tissue. The contracted position also facilitates movement of electrode instrument 300 within the patient's body and removal of electrode instrument 300 from the patient's body. In some embodiments, the entire distal portion 350 of electrode instrument 300, including electrode strip 340, may be retractable into a sheath or shield, for example into a channel 221 of an endoscope 202 (FIGS. 3A and 3B). Retraction of the distal portion 350 of electrode instrument 300 into a sheath may facilitate insertion and navigation of the electrode instrument 300 in a patient by covering the edges of the electrode strip 340 which may otherwise increase friction or catch on surrounding tissue during insertion or navigation. When electrode instrument 300 is carried in working channel 221 of an endoscope and distal portion 350 of electrode instrument 300 is retracted into the endoscope's working channel 221, the endoscope provides a robust vehicle for delivering electrode instrument 300 to the target tissue and for removing electrode instrument 300 from the patient's body.

Shaft 320 is capable of transmitting torque to move second end portion 348 of electrode strip 340 relative to body 310 for deploying electrode strip 340. With reference to FIG. 5, after electrode instrument 300 is positioned in the patient with electrode strip 340 at a treatment site 362 adjacent targeted tissue 364, and with the electrode strip moved outwardly from its sheath, if any, the shaft 320 is then rotated via handle 330 or via a robotic manipulator in a first direction indicated by arrow 366 relative to body 310 for radially expanding the helix 360, to thereby deploy the electrode strip 340 into apposition with targeted tissue 364 at the treatment site. Conversely, the rotation of shaft 320 in the first direction relative to body 310 may be accomplished by rotating body 310 in an opposite direction indicated by arrow 370, relative to shaft 320. In some embodiments (not illustrated), shaft 320 is driven by a motor or other mechanism. In some embodiments, shaft 320 does not extend entirely through body 310 but projects from tip 354 of body 310 and is rotationally driven from within body 310 or by other means. In the embodiment illustrated in FIG. 5, the first direction 366 of rotation of handle 330 is counterclockwise and the relative direction of rotation 370 of body 310 is clockwise for expanding the helix 360. In another embodiment (not illustrated), the direction (i.e., handedness) of the helix may be reversed so that the first direction of rotation of handle 330 is clockwise and the relative direction of rotation of body 310 is counterclockwise.

When in the expanded position or configuration illustrated in FIG. 5, a middle portion 376 of electrode strip 340 between the first and second end portions 346, 348 is spaced apart from shaft 320 and body 310 and into apposition with targeted tissue 364. Once the electrode strip 340 is in contact with targeted tissue 364, electrical potential and/or current may be applied to electrode strip 340 to complete an electrosurgical procedure, as further described below. After completion of the electrosurgical procedure, the electrode strip 340 can be returned to the contracted position (FIG. 4) by rotating shaft 320 relative to body 310 in a second direction opposite first direction 366. Conversely, rotation of shaft 320 in the second direction may be accomplished by rotating body 310 relative to shaft 320 in an opposing second direction opposite from arrow 370. After returning electrode strip 340 to the contracted position, electrode instrument 300 can be moved longitudinally forward or rearward along the treatment site 362 or to a different treatment site, or removed from the patient. In some cases, the electrode strip 340 may be retracted into a sheath, such as into the working channel 221 of an endoscope, before electrode instrument 300 is moved within or removed from the patient.

An expansion ratio of electrode instrument 300 is defined as the diameter of helix 360 when in the expanded position divided by the diameter of helix 360 when in its contracted position. To account for distortions at the first and second end portions 346, 348 of electrode strip 340, the diameter of helix 360 is measured as the diameter of a circle circumscribing an axial projection of the helix 360. Thus, a “maximum expansion ratio” of electrode instrument 300 is defined as the maximum diameter of electrode strip 340 in its maximally expanded position divided by the minimum diameter of electrode strip 340 in its fully contracted position. For clarity, in the maximally expanded position the electrode strip 340 must still extend at least one full rotation or turn (at least 360 degrees), it being understood that less than one full turn or rotation of helix 360 may cause electrode strip 340 to extend radially a greater distance, but primarily or solely from one side of body 310. In some embodiments, the maximum expansion ratio of electrode instrument 300 may exceed 5:1 or may exceed 10:1 or 20:1. In some embodiments, the maximum expansion ratio may be in the range of 5:1 to 30:1. Notably, a usable expansion ratio or “maximum usable expansion ratio” may be somewhat less than the maximum expansion ratio and may depend on mechanical properties of electrode strip 340. For example, a very flexible electrode strip 340 may be tightly wound around body 310 to a very small minimum diameter and when expanded may desirably conform to tissues 362 lining irregular luminal shapes, but when expanded to its maximum diameter the electrode strip 340 may not be able to generate sufficient force in apposition to tissue 362 so as to effect sufficient electrical contact needed to perform a particular electrosurgical function, such as electrolysis and/or electroporation. Conversely, a stiffer electrode strip 340 may be expanded to a greater overall diameter while still providing sufficient apposition force and sufficient electrical contact with tissue for electrosurgical functionality but may be less able to conform to irregular luminal shapes and may be more difficult or impossible to coil tightly around body 310, resulting in a larger minimum diameter in its contracted position. Thus, the term “maximum expansion ratio” should be understood as potentially different from the maximum usable expansion ratio.

In some embodiments, electrode strip 340 comprises a foil strip forming a single electrode of the electrode instrument for monopolar operation. In some embodiments, electrode strip 340 may comprise a flexible printed circuit (FPS) with one or more conductive lines or patterns (e.g., metal layers) exposed on the outer surface of FPS, forming one or more electrodes of the electrode instrument 300. When multiple electrodes are utilized, electrode instrument 300 may be capable of bipolar operation, such as for electrolysis and/or electroporation. In the embodiment illustrated in FIGS. 4 and 5, electrode strip 340 comprises an FPS with two conductors forming two electrodes 380 running the entire length of electrode strip 340. Various conductor shapes and electrode configurations are possible on the surface of FPS. In some embodiments, each electrode 380 is connected to a power source (e.g., power source 12 of FIG. 1) by a wire 388 (FIG. 4) which may extend within shaft and/or body, or along the outside of body 310, for example. When electrodes are implemented in an FPS, the FPS may include a narrow elongate tab or extension having conductive lines extending along the length of body 310 in place of wires, either inside of shaft 320 and/or body 310 or along the outside of body 310. In other embodiments, each electrode 380 is connected to power source 12 via a different conductive pathway, such as via shaft 320 or body 310 (e.g., if made of or including a conductive material). When shaft 320 and/or body 310 forms the conductive pathway for one or more electrodes 380, the shaft 320 and/or body 310 may have an electrically insulated surface to prevent electrical short circuits as between the shaft 320 and body 310, to prevent electrical contact between the body 310 and tissues, and/or to prevent electrical contact or short circuits with electrode strip (e.g., when a foil strip) at unwanted locations beneath helix 360. When shaft 320 and/or body 310 forms the conductive pathway for one or more electrodes 380, in some embodiments the electrical connections to power supply 12 may be achieved through contact pads (not illustrated) formed on proximal end portion 336 of body 310 and/or proximal end portion 332 of shaft 320, and/or in handle 330, which may be electrically coupled to power supply 12 by corresponding contacts, wipers, or other means, such as inductive coupling (not illustrated). In other embodiments, wires 388 may be connected to handle 330 and/or one or both proximal end portions 336 and/or 332 of the respective body 310 and/or shaft 320.

FIG. 4 illustrates electrode strip 340 in the contracted position with at least part of its first end portion 346 closely wrapped around of the distal end portion 342 of body 310 and at least part of its second end portion closely wrapped around the distal end portion 352 of shaft 320 that extends from tip 354 (FIG. 5) of body 310. In some embodiments, all or nearly all of the electrode strip 340 may be helically wrapped around body 310, and in most embodiments at least a small portion of the second end portion 348 of electrode strip 340 is wrapped around the distal end portion 352 of shaft 320 that extends from tip 354 (FIG. 5) of body 310. In most embodiments, the entirety of electrode strip 340 is wrapped around shaft 320, with the distal end portion 342 of body 310 being interposed between the electrode strip 340 and the shaft 320 along at least some of the length of the helix 360 of electrode strip 340. In some embodiments, such as illustrated schematically in FIGS. 6A, 6B and 6C described below, the shaft 320 extends a substantial distance from tip 354 of body 310 and the first end 346 of electrode strip 340 is attached to distal end portion 342 of body at or near tip 354 such that most or all of the electrode strip 340 is wrapped directly around shaft 320. In some embodiments, distal portion 350 of electrode instrument 300 may be smaller than 2 mm in diameter, or smaller than 1.2 mm in diameter, or between 0.8 and 3 mm in diameter, or between 1.2 and 2 mm in diameter. Such a small profile of electrode instrument 300 may facilitate its use for treatment of the duodenum, small intestines, nasal passageways, lung tissue in primary, secondary, or tertiary bronchi or bronchioles, or other narrow luminal regions of a patient's body.

First and second end portions 346, 348 of electrode strip 340 may be attached to the respective body 310 and shaft 320 by various means. In some embodiments, at least one end of electrode strip 340 is attached to body 310 or shaft 320 via an adhesive. In some embodiments, at least one end of electrode strip 340 is welded to body 310 or shaft 320. In some embodiments, at least one end of electrode strip 340 is tacked or pinned to body 310 or shaft 320. In some cases, the attachment means are different for first and second end portions 346, 348, and in some cases the attachment means are the same.

In some embodiments, electrode strip 340 may be heat treated or thermoformed to provide shape memory, preferably in the contracted position. By heat treating or thermoforming electrode strip 340 in the contracted position, electrode strip 340 will tend to return to the contracted position when rotational force is removed from body 310 and/or shaft 320, or in the event that either of the first or second end portions 346, 348 of electrode strip 340 should become detached from body 310 or shaft 320, which may facilitate removal of the instrument from the patient's body.

With reference to FIGS. 4 and 5, electrode strip 340 may be arranged to leave a small gap 394 between adjacent turns of helix 360 when electrode strip 340 is in the contracted position, as illustrated in FIG. 4, but when electrode strip 340 is rotationally deployed the gap increases, as illustrated by the enlarged gaps 396 in FIG. 5. In some embodiments, electrode strip 340 may be arranged to leave no gaps or no substantial gaps between turns of the helix 360 when in in the contracted position. In some embodiments electrode strip 340 is arranged to leave no or little gap between adjacent turns of helix 360 when deployed at the treatment site 362 in the expanded position. When arranged to leave little or no gap between turns of the helix 360 in the expanded position, electrode strip 340 may overlap in the contracted position.

In some embodiments, shaft 320 is slidably movable proximally or inwardly (e.g., retractable) and distally outwardly (e.g., extendable) relative to body 310, and/or body 310 is slidable longitudinally along shaft 320, to adjust a pitch of helix 360 during deployment and/or retraction, as illustrated schematically in FIGS. 6A, 6B, 6C. Such longitudinal sliding movement of shaft 320 may be performed independently of or simultaneously with rotation of shaft 320. FIG. 6A illustrates electrode instrument 400 with a first end of an electrode strip 440 connected to a body 410 and a second end of the electrode strip attached to a shaft 420, and wherein electrode strip 440 is arranged to form a helix 460 and illustrated in FIG. 6A in a contracted position. FIG. 6B illustrates a partially deployed electrode strip 440, wherein the body 410 is rotated relative to shaft 420 and slidably advanced, as indicated by arrows, so shaft 420 is retracted somewhat relative to body 410 as compared to FIG. 6A. And FIG. 6C illustrates electrode strip 440 in an expanded position with body 410 further rotated relative to shaft 420 and slidably advanced along shaft 420. In each case of FIGS. 6A, 6B, and 6C, the pitch of helix 460 may be kept constant by moving body 410 in a distally outward direction relative to shaft 420 (moving shaft 420 inwardly in retraction relative to body 410) as the body 410 and shaft 420 are relatively rotated to expand helix 460. By controlling the pitch of helix 360, 460 by the combination of rotation and sliding inward/outward movement of shaft 320, 420, smaller gaps or no gaps between turns and improved electrical contact with targeted tissue 364 may be achieved in the deployed position, while allowing the electrode to be contracted to a small diameter in the contracted position.

In some cases, the angle of the connection between first and second end portions of electrode strip 340, 440 may be variable by virtue of pivoting connections between the ends of electrode strip 340, 440 and the respective body 310, 410 and shaft 320, 420, so that electrode strip 340, 440 does not buckle when expanded and/or contracted. Such a variable connection angle may instead allow helix 360, 460 to maintain an approximately cylindrical outer profile when shaft 320, 420 is moved distally outwardly or moved inwardly relative to body 310, 410 during respective deployment and retraction.

In various embodiments, deployment and contraction of the electrode strip 340 may be achieved by any combination of sliding (e.g., axial) movement of the shaft 320 and/or body 310 relative to each other and/or rotation relative to each other.

In some embodiments, the shaft 320 and/or body 310 may be slidable (e.g., axially movable) relative to each other for electrode strip 340 deployment/contraction without relative rotation between the shaft 320 and the body 310 (e.g., relative rotation is fixed or prevented). For example, the shaft 320 and/or the body 310 may have a keyed or interlocking relationship that prevents relative rotation between the two, such as one of the shaft 320 and/or the body 310 having a slot and the other having a protrusion or key that slides within the slot but prevents or hinders rotation. As another example, the shaft 320 and/or the body 310 may have corresponding non-circular cross sections (such as multisided shape, such as a triangle, square, rectangle, parallelogram, pentagon, hexagon, etc.) that prevent or hinders relative rotation. In another example, the shaft 320 and/or body 310 may be in the form of a spline shaft (e.g., with ridges or teeth) that allows for relative translation but prevents rotation. As another example, the shaft 320 and/or the body 310 may each have a separately actuatable proximal portions (e.g., handles or the like) that are actuatable by a user and/or by a robotic manipulator when operative coupled to the manipulator. The proximal portions may have interlocking features described above to prevent relative rotation, which may be selectively activated.

In some embodiments, the shaft 320 and/or the body 310 may be rotatable relative to each other for electrode strip 340 deployment/contraction without relative translation between the shaft 320 and the body 310 (e.g., relative axial movement is fixed or prevented). For example, the shaft 320 and/or the body 310 may have mechanical constraints that permit rotation but prevent relative axial movement. As another example, the shaft 320 and/or the body 310 may each have a separately actuatable proximal portions (e.g., handles or the like) that are actuatable by a user and/or by a robotic manipulator when operative coupled to the manipulator. The proximal portions may have interlocking features to prevent relative axial motion, which may be selectively activated.

In some embodiments, the shaft 320 and/or the body 310 may be movable in both relative translation and relative rotation for electrode strip 340 deployment/contraction. For example, the shaft 320 and/or the body 310 may move for relative rotation during a portion of the deployment/contraction and may move in relative translation during another portion of the deployment/contraction. In such embodiments, the shaft 320 and body 310 may be joined by a helical cam slot arrangement or threaded engagement to coordinate the rotational and longitudinal movement. For example, body 310 may include a cam slot and shaft 320 may include a follower that rides in the cam slot.

In embodiments where the electrode instrument 300 is coupled to a robotic manipulator (such as manipulator assembly 102 of FIG. 2) and receives actuation forces from the manipulator, in some embodiments, translation and rotation may be independently driven by separate actuators. That is, the system may include a first actuator to drive rotation and a second actuator to drive translation. A control system may send signals to the respective actuators to drive relative axial motion and/or relative rotation for deployment/contraction of the electrode strip 340. In some examples, the translation and rotation degrees of freedom are independently actuated. During deployment/contraction, both the translation actuator and the rotation actuator may be simultaneously or sequentially actuated to cause deployment and contraction. As one example for sequential actuation, during contraction, the first rotation actuator may be driven to wind down the electrode strip 340 to a preset amount (e.g., wind down from full deployment to 20% deployment, or to 15% deployment, or 25% deployment, etc.), then the second translation actuator may be driven to stretch the helix 360 axially (i.e., change its pitch), then the first rotation actuator may be driven to wind down the remainder of the electrode strip 340 deployment. In some embodiments, the system may include separate actuators for driving translation and rotation with coupled control between the actuators. That is, control of each degree of freedom is coupled such that actuating the rotation actuator for a specified amount of movement will cause the translation actuator to move with a corresponding length. In some examples, the control system may send signals to drive axial motion via the translation actuator without driving rotation or may send signals to drive rotation via the rotation actuator without driving translation. For example, the control system may send signals to the actuator that drives relative axial movement between the shaft 320 and the body 310. The control system may optionally send signals to lock, disengage, or otherwise not drive the actuator that drives relative rotation between the shaft 320 and the body 310. Similarly, the control system may send signals to the actuator that drives relative rotation movement between the shaft 320 and the body 310. The control system may optionally send signals to lock, disengage, or otherwise not drive the actuator that drives relative axial movement between the shaft 320 and the body 310.

In some embodiments where the electrode instrument 300 is coupled to a robotic manipulator and receives actuation forces from the manipulator, one actuator of the manipulator is coupled to drive both translation and rotational movements.

In some embodiments, the robotic manipulator may infer the diameter of helix 360 based on an algorithmic model of the electrode treatment device and the amount of rotational and/or translational actuation of shaft 320 and/or body 310 relative to the other. The amount of rotational and/or translational movement may be determined by encoders on the actuator(s) or inferred by the time and rate at which the actuator(s) drive the electrode instrument 300. In some embodiments, electrical contact of electrode strip 340 with tissue may be detected by measuring resistance across the electrodes 380 of the electrode strip 340. In some embodiments, the electrical inputs to, or electrical characteristics of, the robotic actuators may be monitored to detect a complete deployment of electrode strip 340 into sufficient apposition with tissue. For example, the robotic manipulator may monitor actuator drive torque by measuring the absolute current value(s) and/or the rate of change (slope) of the drive current(s) during rotation and/or translation of the electrode device. In some embodiments, apposition may be detected by sensing that the actuator is drawing current, but not rotating (as may be detected by an encoder on the actuator).

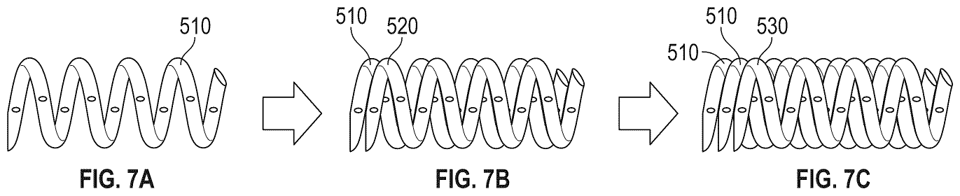

FIGS. 7A, 7B, and 7C schematically illustrate steps in a treatment technique to address the presence of large gaps 396 between turns of the helix 360 when in the deployed position. FIG. 7A illustrates a first contact region or treated region 510 of a helical electrode (not illustrated, but in the same shape as contact region), wherein the treated region is effected by application of electrical potential and/or current to electrode to perform an electrosurgical technique such as electrolysis and/or electroporation. FIG. 7B illustrates a second contact region or treated region 520 that overlaps along its edges with the first contact region or treated region 510. Similarly, FIG. 7C illustrates a third contact region or treated region 530 that overlaps along its edges with the second contact region or treated region 520. Between the treatment steps of FIGS. 7A and 7B, and between the treatment steps of FIGS. 7B and 7C, the electrode strip is contracted, then advanced along the treatment site incrementally by a distance less than the pitch of the helix, then expanded again into apposition with tissue. In the embodiment illustrated in FIGS. 7A-7B-7C, each advancement is approximately ⅓ of the pitch of the deployed helix.

Embodiments of an electrode deployment system are illustrated herein in the context of an electrode instrument 300 for electrosurgery. However, electrode deployment systems consistent with the disclosed embodiments may be utilized for purposes of electrical stimulation, electrical sensing, or as part of other instruments or mechanisms for medical uses, or as parts of instruments or mechanisms for non-medical uses.

Thus, it should be understood that the exemplary embodiments described herein of both the design of catheter delivery systems and the potential clinical applications associated therewith are not intended to be limiting. In addition, it is to be appreciated that any one of the above embodiments or processes, or specific features associated therewith, may be combined with one or more other embodiments and/or processes or be separated and/or performed amongst separate devices or device portions in accordance with the present systems, devices, and methods. Finally, the disclosure is intended to be merely illustrative of the present devices, apparatuses, systems, and methods and should not be construed as limiting the appended claims to any particular embodiment or group of embodiments.

It will be obvious to those having skill in the art that many changes may be made to the details of the above-described embodiments without departing from the underlying principles of the invention. The scope of the present invention should, therefore, be determined only by the following claims.

1-40. (canceled)

41. An electrode treatment device, comprising:
an elongate body having a distal end portion terminating in a tip;
a shaft rotatably supported by the body and extending beyond the tip of the body; and
an electrode strip having a first end portion attached to the body, and a second end portion attached to the shaft at a location distal to the tip of the body, the electrode strip wound around the body and/or the shaft to form a helix, the shaft being rotatable in a first direction relative to the body for radially expanding the helix to thereby deploy the electrode strip at a treatment site, and the shaft being rotatable relative to the body in a second direction opposite the first direction for radially contracting the helix around the body and/or shaft to facilitate extraction of the electrode strip from the treatment site.

42. The electrode treatment device of claim 41, wherein the shaft is slidably movable inwardly and distally outwardly relative to the body.

43. The electrode treatment device of claim 41, wherein the electrode strip comprises a flexible printed circuit.

44. The electrode treatment device of claim 41, wherein the body or the shaft, or both, provide a conductive path to the electrode strip, and wherein the body or the shaft, or both, have an electrically insulated surface.

45. The electrode treatment device of claim 41, wherein the electrode strip is arranged to leave no gaps between turns of the helix when deployed at the treatment site.

46. The electrode treatment device of claim 41, wherein the electrode strip is arranged to leave no gaps between turns of the helix when the electrode strip is contracted around the body and/or the shaft.

47. The electrode treatment device of claim 41, wherein the electrode strip is thermoformed or heat treated to return to the shape of the helix when no force is applied to the electrode strip via the shaft and/or the body.

48. The electrode treatment device of claim 41, further comprising a robotic manipulator coupled to the shaft and/or the body, and wherein the robotic manipulator includes a rotational actuator that rotates the shaft relative to the body and a translational actuator that moves the shaft axially relative to the body.

49. The electrode treatment device of claim 48, wherein the rotational actuator and translational actuator are integrated in a single actuator device.

50. The electrode treatment device of claim 48, further comprising a control device that controls the rotational and translational actuators independently.

51. The electrode treatment device of claim 48, wherein a deployment control device detects when the electrode strip is deployed into apposition with targeted tissue by sensing an electrical input and/or characteristic of at least one of the rotational and translational actuators.

52. An electrode treatment device comprising:
an elongate body having a distal end portion terminating in a tip;
a shaft extending from the tip of the body and movable relative to the body; and
an electrode strip having a first end portion attached to the body, a second end portion attached to the shaft at a location distal to the tip of the body, and a middle portion between the first and second end portions, the electrode strip being coiled around the shaft or the body, or both, and the shaft being movable relative to the body to cause the electrode strip to move between a contracted position in which the electrode strip is wrapped closely around the shaft or the body, or both, and a maximally expanded position in which the middle portion of the electrode strip is spaced apart from the shaft and the body, wherein the electrode strip has a maximum expansion ratio that exceeds 5:1.

53. The electrode treatment device of claim 52, wherein the shaft is rotatable relative to the body.

54. The electrode treatment device of claim 52, wherein the shaft is slidably movable inwardly and distally outwardly relative to the body.

55. The electrode treatment device of claim 52, wherein the electrode strip comprises a flexible printed circuit.

56. The electrode treatment device of claim 52, wherein the body or the shaft, or both, provide a conductive path to the electrode strip, and wherein the body or the shaft, or both, have an electrically insulated surface.

57. The electrode treatment device of claim 52, wherein the maximum expansion ratio exceeds 10:1.

58. The electrode treatment device of claim 52, wherein the expansion ratio is in the range of 5:1 to 30:1.

59. The electrode treatment device of claim 52, wherein the electrode strip is helically coiled around the shaft or the body, or both.

60. The electrode treatment device of claim 59, wherein the electrode strip is thermoformed or heat treated to return to the contracted position of the helix when no force is applied to the electrode strip via the shaft and/or the body.

61. The electrode treatment device of claim 52, further comprising a robotic manipulator coupled to the shaft and/or the body, and wherein the robotic manipulator includes a rotational actuator that rotates the shaft relative to the body and a translational actuator that moves the shaft axially relative to the body.

62. The electrode treatment device of claim 61, wherein the rotational actuator and translational actuator are integrated in a single actuator device.

63. The electrode treatment device of claim 61, further comprising a control device that controls the rotational and translational actuators independently.

64. The electrode treatment device of claim 61, wherein a deployment control device detects when the electrode strip is deployed into apposition with targeted tissue by sensing an electrical input and/or characteristic of at least one of the rotational and translational actuators.
